(19) United States
(12) Patent Application Publication
(10) Pub. No.: US 2005/0177087 A1
(43) Pub. Date: Aug. 11, 2005

(54) Blood heat conserving tube assembly for hemodialysis or blood transfusion

(76) Inventor: Steve Lee, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/775,111

(22) Filed: Feb. 11, 2004

(51) Int. Cl. A61M 37/00

(52) U.S. Cl. 604/6.13

(57) ABSTRACT

A blood heat conserving tube assembly includes a mediate inner tube for transporting blood, an outer tube surrounding the mediate inner tube and connected by several ribs. Channels are formed between the inner and the outer tubes. A hollow connector set and a space is within. Several hollow spaces are connected with extension pieces. Each extension piece is inserted between the inner and out tube channel; thus, the space and its channel are connected. Through these open space and channels flow the fluid with a proper temperature heats up the supplying blood temperature.

### BACKGROUND OF THE INVENTION

1. Field of the Invention

The present invention relates to a BLOOD HEAT CONSERVING TUBE ASSEMBLY, particularly to the blood heat conserving for a patient undergo blood transfusion or hemodialysis process. The device maintains the blood heat during the external transportation and keeps the patients from hypothermia.

### ILLUSTRATION OF THIS INVENTION

### 1. THE TECHNICAL SCOPE OF THIS INVENTION

This invention of BLOOD HEAT CONSERVING TUBE ASSEMBLY is mainly designed to maintain the temperature of blood supply to a certain degree during the external transportation to keep patients warm during blood transfusion or hemodialysis without causing hypothermia to the patients.

### 2. INTENTION OF PRECEDENT TECHNOLOGY AND THIS INVENTION

Either having a hemodialysis performed in a large-scaled hospital or a blood transfusion in a medical clinic, the blood supply needs to be heated up before it is transfused to the patients. The body temperature drops because of lack of devices to maintain the temperature of heated blood supply before it reaches to the patient. The temperature of blood supply is usually much lower than that of human body. Thus a blanket is applied to keep patient warm and maintain a comfy position. Or patients may suffer from hypothermia or death to some extent.

However, by only covering a blanket to maintain body temperature in an air-conditioned room is not a good solution to cope with hypothermia problem.

Therefore, to increase comfort and prevent hypothermia for patients as well as to minimize heavy workload for nursing staffs are the intention of this invention.

### 3. THE CONTENT OF THIS INVENTION

The main purpose of this innovation is to provide two decks of hollow tube assemblies to transport blood supply through the channel of inner tube; and a heated fluid or air through the channel of outer tube to keep blood supply maintaining at an appropriate body temperature. Thus, patients are safeguarded against hypothermia.

Other objects, advantages and novel features of this invention will be more apparent from the following detailed description when taken in conjunction with the accompanying drawings.

### 4. BRIEF DESCRIPTION OF THE DRAWINGS

FIG. 1 is an exploded perspective view of the blood heat conserving tube assembly of the present invention;

FIG. 2 is a longitudinal cross section view of the assembled blood heat conserving tube assembly;

FIG. 3 is a schematic side plan view showing a distal end of the mediate tube;

FIG. 4 is a schematic side plan view showing the interrelationship between the distal end of the mediate tube and the connector tube; and

FIG. 5 is a perspective view of the blood heat conserving tube assembly of the present invention.

### 4. DETAILED DESCRIPTION OF APPLICATION

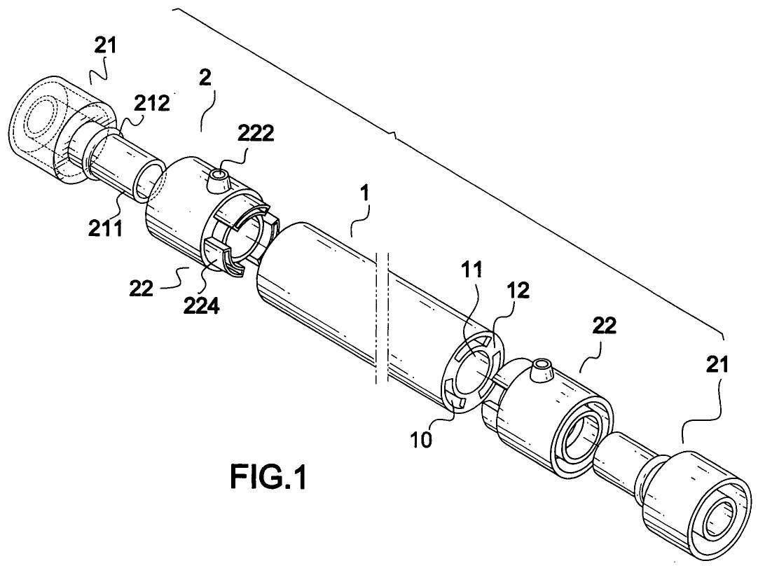

Please refer to FIG. 1, this invention of blood heat conserving tube assembly consists of one mediate tube (1) and two connector sets (2) that connect to each end of the mediate tube respectively.

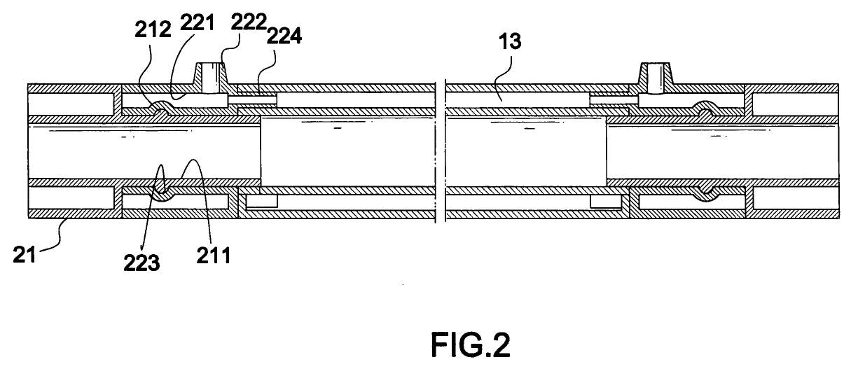

There is an inner tube (11) inside the outer mediate tube (1). The inner tube (11) and the outer mediate tube (1) are connected by ribs (12) extending between the outer periphery of the inner tube (11) and the inner periphery of the mediate tube (1), which has formed many channels (13) between the inner tube (11) and the outer mediate (1) tube. Due to the formation of the ribs (12), channels (13) are defined between two adjacent ribs (12) and which has formed many openings (10) around each end of inner and outer mediate tubes (11,1) as shown in FIG. 2.

Because the two connector sets (2) are substantially the same in structure, the following description is focused on only one connector set (2) for brevity.

Each connector set (2) has a connecting head (21) and an engaging tube (22) located between connecting head (21) and inner and outer mediate tubes (11, 1).

Please refer to FIG. 2. The above-mentioned connecting head (21) actually is a hollow tube. A connecting tube (211) is extended from the middle of connecting head (21). In the meantime, a circle of flange (212) is formed on the outer surface of connecting tube (211).

There is a space (221) inside the engaging tube (22). An intlet (222) is opened on the outer surface of engaging tube (22) and connects to the space (221). An annular groove (223) located at one side of inner wall of engaging tube (22) corresponds to its counterpart—flange (212) of connector head (21); several open spaces extended from the other side of engaging tube correspond to their counterpart—extension pieces (224) of openings (10).

When all the parts of this invention are assembled, the completed structure of BLOOD HEAT CONSERVING TUBE ASSEMBLY is exactly like the picture shown in FIGS. 2, 3, 4, and 5. Each extension piece is hollow. When extension piece (224) is inserted into its opening (10), the space (221) inside the engaging tube (22) is connected to the channel (13); because of their counterpart relationship, annular groove (223) and flange (212), connecting head (21) and engaging tube (22) are firmly connected. Since connecting head (21), engaging tube (22) and inner tube (11) are hollow; a passage is formed through these hollow spaces, the blood supply of inner tube (11) successfully runs through.

It is to be noted that, when the assembly of this invention is completed, the space (221) and its channel (13) are connected and surround the blood supply of inner tube (11), the operator should instill fluid, such as air or water, at a specified temperature into the inlet (222). The heat from fluid has a function of increasing and conserving the heat of blood supply. Thus, the temperature is maintained at a steady level. Under this situation, patients receiving blood transfusion won't be endangered under hypothermia.

The variety of fluid adopted in this invention of BLOOD HEAT CONSERVING TUBE ASSEMBLY is quite flexible. Besides, the construction of this invention is very simple, thus, the manufacturing cost reduced substantially.

### 5. BRIEF ILLUSTRATION OF FIGURES

Figure Part

FIG. 1: A three-dimensional dissected view of this innovation

FIG. 2: A longitudinal cross section view of this innovation

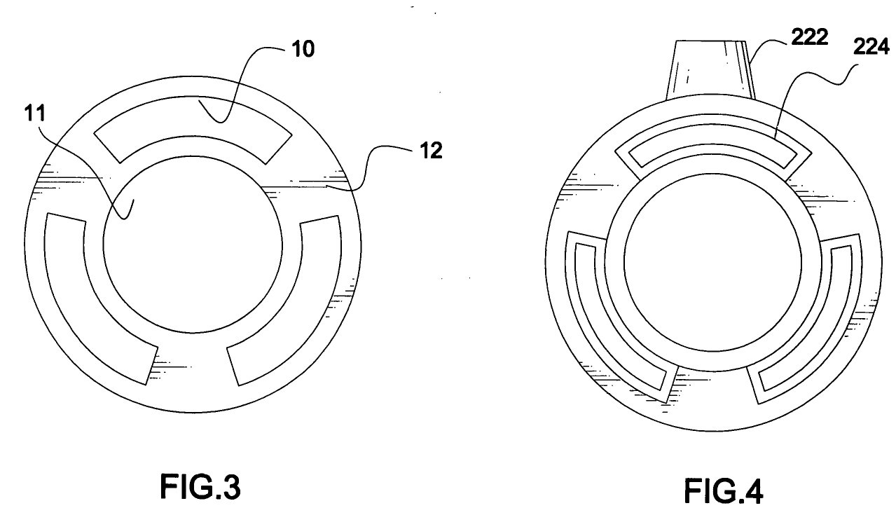

FIG. 3: A symmetric view of inner and outer tube in this innovation

FIG. 4: A plane figure of an inserted extension piece of engaging tube

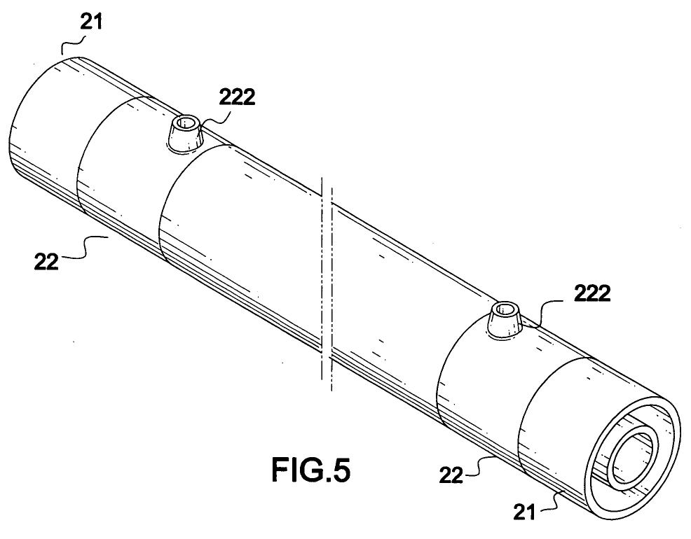

FIG. 5: A three-dimensional view of an assembled Blood Heat conserving Tube Assembly

Symbols

| | | | |
|---|---|---|---|
| (1) | outer mediate tube | (10) | opening |
| (11) | inner tube | (12) | rib |
| (13) | channel | (2) | connector set |
| (21) | connecting head | (211) | connecting tube |
| (212) | flange | (22) | engaging tube |
| (221) | space | (222) | intlet or outlet |
| (223) | annular groove | (224) | extension piece |

1. A blood heat conserving tube assembly, it consists:
A mediate inner tube transports blood supply.
An outer tube surrounds the mediate inner tube, and connected by several ribs. Thus, a few channels are formed between the inner and the outer tube.
A hollow connector set and a space is within. Several hollow spaces are connected with extension pieces. Each extension piece is inserted between the inner and out tube channel; thus, the space and its channel are connected.
Through these open space and channels flow the fluid with a proper temperature heats up the supplying blood temperature.

2. As above-mentioned assembly (1.), its hollow connector set consists of:
A hollow connecting head, and a connecting tube is extended at one side; and
a hollow engaging tube is connected with its connecting head, thus, a passage is formed within.

3. As above-mentioned assembly (2.), its extension piece is formed at another side of engaging tube.

4. As above-mentioned assembly (1.), an inlet is connected with connecting head. A specified temperature of fluid is instilled into this inlet and flows through the spaces and channels with the function of heating up the blood supply.

5. As above-mentioned assembly (3.), an intlet is connected with engaging tube. A specified temperature fluid is instilled into and flows through the spaces and channels with function of heating up the blood supply flows out from the outlet of the other end.
